(19) United States
(12) Patent Application Publication
(10) Pub. No.: US 2017/0143650 A1
(43) Pub. Date: May 25, 2017

(54) TREATMENT OF A SKIN LESION

(71) Applicant: MEDICAL BRANDS RESEARCH B.V., Amsterdam (NL)

(72) Inventor: Maikel HENDRIKS, Amsterdam (NL)

(21) Appl. No.: 15/316,414

(22) PCT Filed: Jun. 5, 2015
(86) PCT No.: PCT/EP2015/062613

§ 371 Date: Dec. 5, 2016

(30) Foreign Application Priority Data
Jun. 5, 2014 (EP) 14171405.5

(51) Int. Cl. A61K 31/19; A61K 47/08; A61M 11/00; A61K 41/00; A61M 35/00; A61K 9/00; A61K 31/07

(52) U.S. Cl. CPC A61K 31/19; A61K 9/0014; A61K 47/08; A61K 31/07; A61K 41/0052; A61M 35/003; A61M 11/007; A61M 2205/3337

(57) ABSTRACT

The invention provides a spray applicator (1) for topical treatment, the spray applicator (1) comprising a container (2) comprising a treatment liquid (52) under pressure and a propellant (51), wherein the container (2) is in fluid connection with a spray valve system (4) for application of at least part of the treatment liquid (52) on a topical area, wherein the treatment liquid (52) comprises an acid, wherein the container (2) comprises a container wall (20) having a top layer (22) and a top layer coating (21) on said top layer (22), wherein the top layer (22) optionally comprises a metal and wherein the top layer coating (21) comprises a polymer and/or a resin.

### FIELD OF THE INVENTION

The invention relates to a spray applicator, the use of such spray applicator and the use of the treatment liquid (that is contained in the spray applicator). The invention further relates to a composition comprising said treatment liquid for cosmetical and/or medical use. Further the invention relates to a method for providing said spray applicator.

### BACKGROUND OF THE INVENTION

Various compositions are known for the treatment of skin lesions, such as warts, corn and calluses, actinic keratosis, keratosis pilaris, acne, skin hyperpigmentation and/or nail lesions, such as ingrown toenails and/or lesions in mucous membranes, such as cold sores and mouth ulcers. WO2012007584, for instance, describes a composition for the treatment of superficial lesions, in particular skin lesions, mucous membrane lesions and/or nail lesions, an applicator comprising such a composition and the use of such a composition. The composition comprises an effective amount of trichloroacetic acid, at least one thickener, and at a physiologically acceptable solvent. The composition is considered effective against a plethora of superficial lesions selected from the group consisting of viral warts, verrucae, water warts (molluscum contagiosum), corns and calluses, and skin hyperpigmentation: age spots, solar lentigo, senial lentigo, acne, keratosis pilaris, actinic keratosis, mouth ulcers (canker sores), cold sores, ingrown toenails, onychomycosis, eyelid xanthelasma.

WO2007139378 describes a device for cold treating a tissue. The invention also relates to an assembly of such a device and a container comprising a cryogenic liquid, such as a spray can. The invention further relates to a method for cold treating a tissue.

WO2012007584 describes a composition for the treatment of superficial lesions, in particular skin lesions, mucous membrane lesions and/or nail lesions, an applicator comprising such a composition and the use of such a composition. The composition comprises an effective amount of trichloroacetic acid, at least one thickener, and at a physiologically acceptable solvent. And is effective against a plethora of superficial lesions selected from the group consisting of viral warts, verrucae, water warts (molluscum contagiosum), corns and calluses, and skin hyperpigmentation: age spots, solar lentigo, senial lentigo, acne, keratosis pilaris, actinic keratosis, mouth ulcers (canker sores), cold sores, ingrown toenails, onychomycosis, eyelid xanthelasma.

### SUMMARY OF THE INVENTION

Many prior art method to treat warts or other skin lesions are not effective or not effective enough. Further, some of the prior art methods need specific care and medical supervision, such as the treatment of warts with liquid nitrogen.

Hence, it is an aspect of the invention to provide an alternative method and/or applicator, which preferably further at least partly obviate one or more of above-described drawbacks, and which is especially safe.

In a first aspect, the invention provides a spray applicator for topical treatment, the spray applicator comprising a container comprising a treatment liquid under pressure and a propellant, wherein the container is in fluid connection with a spray valve system, especially a dosage controlled spray valve system, for application of at least part of the treatment liquid on a body surface (herein also indicated as topical area), wherein the treatment liquid comprises an acid, wherein the container comprises a container wall having a top layer and optionally a top layer coating on said top layer. In a specific embodiment, the top layer comprises a metal and the top layer coating comprises in an embodiment a polymer, such as polypropylene or polyethylene, and/or a resin. In yet a further aspect, the invention provides a spray applicator, especially for topical treatment, the spray applicator comprising a container comprising a treatment liquid under pressure and a propellant, wherein the container is in fluid connection with a spray valve system (also comprised by the spray applicator) for application of at least part of the treatment liquid on a topical area, wherein the treatment liquid comprises an acid, wherein the container comprises a container wall which may comprise aluminum or (stainless) steel and a coating comprising a polymer and/or a resin, wherein the spray applicator further especially comprises a distance holder configured to arrange on a topical area and comprising a opening configured to enclose a topical lesion, wherein the spray applicator is configured to spray the treatment liquid through the opening (herein also indicated as “first opening”) in the distance holder, especially thereby reducing exposure of topical areas that need not be exposed to the treatment liquid. At least part of such (healthy) topical area is covered by part of the distance holder (the part that surrounds the opening in the distance holder).

Hence, in an embodiment the container comprises a container wall comprising aluminum or (stainless) steel and a coating comprising one or more of a polymer and a resin, wherein the top layer coating comprises one or more of polyethylene and polypropylene, and wherein the metal of the top layer of the wall (thus) comprises aluminum or (stainless) steel.

The invention also provides such composition per se, i.e. a composition comprising such treatment liquid and the propellant as defined herein, especially for use in freezing and acid treatment of a topological lesion, such as in a cosmetic treatment. The invention also provides a composition comprising such treatment liquid, especially for use in (acid) treatment of a topological lesion, such as in a cosmetic treatment. The invention also provides such composition comprising such treatment liquid and the propellant according as defined herein, especially for use in freezing and acid treatment of a topological lesion, such as in a medical treatment. The invention further provides such composition especially for use in the treatment of a wart (or corn). Further, the invention also provides such treatment liquid per se, especially for use in freezing and acid treatment of a topological lesion, such as in a cosmetic treatment. The invention also provides such treatment liquid, especially for use in freezing and acid treatment of a topological lesion, such as in a medical treatment. The invention further provides such treatment liquid especially for use in the treatment of a wart (or corn). In a specific embodiment, the composition may further comprise vitamin A. In yet a further embodiment, the treatment liquid further comprises vitamin A.

The acid in the treatment liquid is especially applied to peel the skin or mucous membrane, especially the skin, by which the skin or mucous membrane may renew itself. The propellant is not only used to provide the spray, but may especially advantageously also cool the treatment liquid and/or the topical lesion. Furthermore the propellant is also supporting a penetrating effect into the epidermis of the skin to provide a more in-depth penetration of the treatment liquid. The treatment liquid and the propellant may both have an advantageous effect on the lesion to be treated, such as a wart (or corn): freezing, due to the propellant, may induce blister formation and the acid may have a peeling effect. This may thus lead to a freezing effect, in addition to the acid treatment or acid effect. It appears that this combined treatment is more efficient than the separate treatments alone. Further, the invention may allow a more effective treatment than conventional cryotherapy but may be more efficient than traditional wart acid treatments. The treatment liquid, especially the acid, may provide an oxidation effect to the lesion.

The spray applicator or the treatment liquid, or the treatment liquid in combination with the propellant, may be used for several types of lesions. The spray applicator, or the treatment liquid, or the treatment liquid in combination with the propellant, may be applied for medical use and/or non-medical use, such as cosmetical use. Herein, the term “treatment liquid” refers to the liquid comprising the acid and optionally one or more other components, and the term “treatment composition” refers to the composition or mixture comprising the treatment liquid and the propellant, which is applied on the topical area (and which escapes as spray yet from the spray valve system).

In a further aspect, the invention especially provides the use of the spray applicator, as defined herein, for a (cosmetical) treatment of a topical lesion selected from the group consisting of epidermodysplasia veruciformis, a HPV (human papillomavirus) caused skin disorder, acne, a scar, a wrinkle, a tattoo, eyelid xanthelasma, an age spot, a senial lentigo, a solar lentigo, a melasma, a hand wart, a plantar wart, a corn, an ingrown toe nail, a mucous membrane lesion. The term “wart” may e.g. relate to one or more of verruca vulgaris and verruca plantaris. The treatment liquid is especially suitable for the treatment of one or more of a wart and a corn. Likewise, also the treatment liquid per se may be used. Hence, the invention provides in yet a further aspect also the use of the treatment liquid as defined herein, for a cosmetical treatment (or a medical treatment) of a topical lesion selected from the group consisting of epidermodysplasia veruciformis, a HPV caused skin disorder, acne, a scar, a wrinkle, a tattoo, eyelid xanthelasma, an age spot, a senial lentigo, a solar lentigo, a melasma, a hand wart, a plantar wart, a corn, an ingrown toe nail, a mucous membrane lesion.

The term topical treatment especially relates to the treatment with the treatment liquid (and propellant) to a body surface, such as the skin or a mucous membrane, especially the (human) skin.

In a specific embodiment, the spray applicator one or more of the following conditions, especially all, may apply: (i) the acid comprises one or more acids selected from the group consisting of trichloro acetic acid, salicylic acid, formic acid, glycolic acid, dichloro acetic acid, monochloro acetic acid, boric acid, nitric acid, sulfuric acid, phosphoric acid, oxalic acid, lactic acid, hydrochloric acid; (ii) the propellant comprises one or more propellants selected from the group consisting of dimethyl ether (DME), diethyl ether (DEE), methyl ethyl ether (methoxy ethane), carbon dioxide (CO₂), nitrogen (N₂), nitrous oxide, propane, butane, liquified petroleum gases (LPG), and a chlorofluorcarbon (CFC); (iii) the top layer coating comprises a polyolefin, especially one or more of polyethylene and polypropylene and/or a resin; and (iv) wherein the metal of the top layer comprises aluminum or (stainless) steel or (stainless) steel. Even more especially, the treatment liquid comprises at least trichloroacetic acid (TCA) and wherein the propellant comprises at least one or more of dimethyl ether (DME), diethyl ether (DEE), and methyl ethyl ether (methoxy ethane). Hence, the acid may also comprise a combination of two or more different acids. A chlorofluorcarbon (CFC) may e.g. be selected from the group consisting of dichlorodifluoromethane (CCl₂F₂) and chlorodifluoromethane (CHClF₂). Further, also the propellant may comprise a combination of two or more propellants.

The top layer coating, when available, or the top layer, in case no coating is available, especially comprises an acid resistant material. Especially good materials for the top layer coating may be selected from the group consisting of chlorinated polyvinyl chloride (CPVC), ethylene chlorotrifluoroethylene (ECTFE), (enhanced) polytetrafluoroethylene, (enhanced PTFE), high-density poly ethylene (HDPE), polyether ether ketone (PEEK), polypropylene (PP), polysulfone (PSU), polyphenylene sulfide (PPS), polyvinyl chloride (especially type 1 (PVC, Type 1) or type 2 (PVC, Type 2)), polyvinylidene fluoride (PVDF), polytetrafluoroethylene (PTFE), Polyamide-imide (PAI), ultra high molecular weight polyethylene (UHMW). These materials are especially resistant against the acid content of the container. These materials may however also be used as wall of the container (and thus the top layer comprising such material).

The coating(s) (or internal lining(s)) which can be used in combination with or stand alone with the coating may be selected from the group of an epoxy phenolic, a polyamidimid, a polyester and a vinyl organosol.

Especially, the propellant has a boiling point of less than 10° C., or even less than −20° C. Further, in a specific embodiment the pressure in the container is in the range of 1.5-80 bar, like 1.5-60 bar, such as especially 1.5-10 bar, like 4-10 bar, such as especially at least 5 bar.

Alternatively, the propellant, or optionally a further component of the treatment liquid, may have a heating effect (on the topical area (such as the skin) to which the treatment composition is applied. Both a propellant having a cooling effect and a propellant having a heating effect (or a further component having such effect), may lead to blister formation. Hence, optionally an exothermic effect on the topical area may be achieved. This (also) facilitates e.g. the removal process of the wart (or corn). For instance, after the application of the treatment liquid, after some time the wart may fall off.

Of course, the treatment liquid may optionally comprise one or more other ingredients, including one or more other active ingredients. The composition as described herein may comprise other ingredients commonly used in cosmetics and pharmaceutical products, such as one or more of a surfactant, a colorant and a perfume.

Trichloroacetic acid (TCA) in the treatment liquid as described herein, proved to be effective against a plethora of skin lesions, in particular warts, corn and calluses, molluscum contagiosum, acne, skin hyperpigmentation, actinic keratosis as well as nail lesions including ingrown toenails and onchyomycosis, and lesions in mucous membranes such as mouth ulcers and cold sores. Application of trichloroacetic acid is particularly effective against genital warts, and especially against ano-genital warts where it has been studied in comparative trials where its effectiveness was compared with other anti-wart therapies. In addition, TCA application was effective various other skin disorders, in particular: epidermodysplasia veruciformis- skin disorder also caused by HPV virus, acne, to remove acne scars and wrinkles, tattoo removal, eyelid xanthelasma, age spots, senial lentigo and solar lentigo, melasma.

In a specific embodiment, the acid comprises one or more acids selected from the group consisting of trichloro acetic acid, salicylic acid, glycolic acid, dichloro acetic acid, monochloro acetic acid, boric acid, nitric acid, sulfuric acid, phosphoric acid, oxalic acid, and lactic acid.

Additionally or alternatively, the treatment liquid may comprise one or more of formic acid and salicylic acid, which alone or in combination may also provide a good result. Hence, in an embodiment the treatment liquid comprises TCA, and optionally formic acid, and in another embodiment the treatment liquid comprises formic acid, and optionally TCA.

Salicylic acid has a positive effect in treating skin and nails. In addition to that, salicylic acid gives a mild anaesthetic effect. Salicylic acid in combination with TCA showed a synergistic effect against skin lesions and nail lesions. Another advantage is that using a combination of salicylic acid and TCA allows for a composition with a relatively low concentration of each of salicylic acid and TCA with a similar effect to compositions using only TCA or only salicylic acid. Using the relatively low concentrations of TCA and salicylic acid decreases the chance of skin irritation due to either of these compounds. Moreover, salicylic acid diminished the discomforting burning feeling on skin and nails sometimes experienced by persons treated with products containing substantial amounts of TCA.

The treatment liquid may thus amongst others be used in for instance a skin peeling treatment, for the medical or cosmetic treatment of skin lesion selected from the group consisting of warts, corn and calluses, and for nail treatments including ingrown toenails. A postulated mechanism of action is that the composition comprising TCA, or another acid, softens the skin or nail, and enables to peel the skin or nail lesion away. For severe lesions, multiple treatments may be needed. Both cosmetic and medical treatments may be performed with the compositions according to the invention. Other active ingredients contributing to the treatment of the lesion may be added. For instance salicylic acid (see also above) is another component effective in corroding the skin or nails, and may be used as an additional active ingredient in combination with trichloroacetic acid. The freezing effect of the propellant seems to enhance these mechanisms, or at least the peeling mechanism, as it facilitates blister formation. Hence, the invention further provides the use of a treatment liquid for the cosmetic treatment of the human skin. Especially, the composition is used for a skin peeling treatment or the treatment of a skin lesion selected from the group consisting of warts, corn and calluses. The compositions are particularly effective against warts and related lesions, including viral warts, verrucae, and water warts (molluscum contagiosum). These treatments are typically considered cosmetic treatments rather than medical treatments. The invention also provides the use of a composition as described herein for the cosmetic treatment of nails. In particular nail deformations (for instance as a result from onchyomycosis) and ingrown toenails may be treated effectively.

The invention further provides the use of a treatment liquid or treatment composition as described herein for the treatment of lesions in mucous membranes, in particular mouth ulcers and cold sores. The invention further provides the use of the treatment liquid or treatment composition as described herein for the cosmetic treatment of superficial lesions selected from the group consisting of viral warts, verrucae, water warts (molluscum contagiosum), corns and calluses, and age spots, solar lentigo, senial lentigo, acne, mouth ulcers (canker sores), cold sores ingrown toenails

The treatment liquid especially comprises an aqueous liquid, such as especially water, and the acid. Especially, the total amount of other components in the treatment liquid, other than the acid and the liquid, such as the aqueous liquid, is less than 10 wt. %, especially less than 5 wt. %, examples of other compounds, such as a colorant or a perfume, are mentioned above. Especially, the treatment liquid comprises the acid in a concentration up to 40 wt. %, such as at maximum 35 wt. %, even more especially at maximum 20 wt. %. Higher acid concentrations may lead to undesired topological effects and/or may be unsafe, such as in the kind of application with children or specific topical parts, like mucous membrane. In a specific embodiment, the treatment liquid comprises the acid in a concentration in the range of 1-10 wt. %. Lower concentrations than 1 wt. % may not be effective enough. Especially, the concentration of the acid in the treatment liquid is at least 2 wt. %, even more especially at least 3 wt. %.

The spray applicator allows without contact of the spray valve system of the applicator with the skin, etc., the treatment of the topological lesion. This is safer and also more hygienic than applicators that need contact with the lesion. The spray applicator may especially be configured to provide a single spray dose of in embodiments not more than 1 ml, such as especially not more than 0.75 ml, such as 0.1-0.5 ml, like 0.1-0.2 ml, or less than 0.2 ml. As will be known by the person skilled in the art, this can be tuned by amongst others the nozzle (outlet) dimensions and the pressure of the propellant in the container. A dosage valve or dosage application may be used to control that the desired spray dose is substantially always met. In some instances, especially in view of safety, a narrow spray may be desirable. Hence, in an embodiment the spray applicator is configured to provide a spray having a spray angle (opening angle) (θ) wherein 90% of a spray dose escapes from an outlet of the spray valve system, wherein the spray angle (θ) is in the range of 15-45°. The spray angle may be tuned by the dimensions of the nozzle. For instance, the nozzle (of the spray valve system) may comprise a conical channel.

The applicator is especially configured to provide a pressurized liquid to the skin. The applicator is especially configured to provide a fine stream of pressurized liquid. A high-speed stream of fluid may impact the skin lesion and delivers cold and acid. This pressurized liquid penetrates better into the skin than a non-pressurized. Hence, with the present invention a combination of a cryogenic effect, a peeling effect, and a penetration effect maybe obtained. This appears to provide better results than a single effect or a subsequent application of the effects. Temporarily, the skin may cool down to a temperature below 0° C., even below −5° C., such as even down to about −90° C. Hence, in an embodiment the applicator is configured to provide the treatment liquid to the skin and induce a temporarily cooling of the skin at the topical area where the liquid is applied to a temperature below −5° C., such as below −20° C., like in the range of −5-−50° C. Furthermore the pressure that is provided by the container can also have beneficial effects due to the fact that the treatment solution is provided deeper in the epidermis. Hence a pressured container is provided, that allows a deeper skin penetration of the treatment liquid than conventional applications and applicators.

In view of safety, especially child safety, the spray valve system may in an embodiment include an infant opening protection. For instance, infant opening protection system may include a turning system that has a state wherein the spray valve system is blocked and a state wherein the spray valve system can be applied.

The container comprises a container wall. The container comprises a top layer or top surface, i.e. the outer part of the wall, which is in physical contact with the treatment liquid and/or propellant when the container is filled. This wall may be entirely of a metal such as aluminum or (stainless) steel. In such instance, the top layer is by definition aluminum or (stainless) steel. Hence, the container wall may be made of aluminum or (stainless) steel. On top of this top layer, especially in view of life time of the spray applicator, the container wall comprises a coating. It appears that a polymer coating, especially selected from the group consisting of polypropylene and polyethylene provide good properties. The coating may include a single-layer coating or a multi-layer coating. In case of the latter coating, adjacent layers of the multi-layer coating may differ in composition and/or thickness. The polypropylene polymer may e.g. be selected from the type of homopolymer, random copolymer, and block copolymer. The comonomer is typically used with ethylene. Likewise, the polyethylene polymer may e.g. be selected from the type of homopolymer, random copolymer, and block copolymer. Examples of PE may e.g. be selected from the group consisting of ultra-high-molecular-weight polyethylene (UHMWPE), high-density polyethylene (HDPE), cross-linked polyethylene (PEX or XLPE), medium-density polyethylene (MDPE), linear low-density polyethylene (LLDPE), low-density polyethylene (LDPE), and very-low-density polyethylene (VLDPE). Hence, the container wall may comprise a metal (layer), especially aluminum or (stainless) steel, and an inner surface coating comprising a polymer coating. However, alternatively, the container wall may comprise a polymer and/or resin, such as one of the above indicated polymers, or one or more of the above indicated acid resistant polymers. In a further embodiment, the container wall comprises a polymer and a polymer and/or resin top coating, such as one or more of the above indicated acid resistant polymers.

In a further embodiment, the spray applicator may comprise a distance holder configured to arrange on a topical area, such as the skin, wherein the distance holder comprises an opening configured to enclose a topical lesion. The user may thus arrange the spray applicator on the skin with the opening surrounding the skin lesion. In this way, exposure of healthy skin to the treatment liquid may be minimized, but hereby it may also be prevented that the spray valve system comes into contact with the topical lesion (such contact is not desirable). The distance holder is an element that can be used to arrange the spray applicator to a topical area with a (more) controlled distance from the spray valve system to the topical lesion to be sprayed with the treatment liquid. In this way, the dosage the lesion receives can better controlled than with a manual arrangement of the spray applicator over the lesion, in addition the distance will also contribute to the effectiveness since the mixture needs to vaporize in order to have a cooling effect. Hence, especially the spray applicator is configured to spray the treatment liquid through the opening in the distance holder. Further, the distance holder is configured to prevent physical contact between the spray valve system and the topical area. Optionally, the opening of the distance holder is adjustable in size. For instance, the distance holder may include an adjustable diaphragm (as opening). In this way, even more the healthy topical area may be protected and efficient and targeted application of treatment liquid to the desired skin lesion is ensured. The distance holder may be integrated in the spray applicator, or may be an attachable unit, e.g. with Snap-On snap-off elements. The distance holder may include a transparent part, that facilitates visual inspection by a user of the area that is sprayed. Hence, the applicator may be designed to ensure a visual control during the use of the applicator to ensure a safe and effective treatment. Alternatively or additionally, the distance holder may be configured in such a way that a user may visual inspect the area that is sprayed when used by the user himself or herself. The opening in the distance holder may be round or square or oval, or have another shape. Especially, part of the distance holder is configured to fit on a human body. Optionally, a plurality of distance holders may be provided, which may fit to different parts of the body.

The transparent part may include one or more of a transparent material and an opening. In an embodiment, the distance holder has a conical shape. In yet another embodiment, the distance holder has a cylindrical shape. In an embodiment, the distance holder is comprises a hollow body, with a wall of the body configured for instance to provide the conical or cylindrical shape, with the wall comprising the transparent part. The wall may be entirely of a transparent material. However, the wall may also include one or more openings. Such openings may be used for one or more of visual inspection and influx of air, entrained with the spray. This influx of air also appears to have a beneficial effect on especially the peeling effect of the acid. Further, the distance holder is especially configured to protect the healthy skin surrounding the topical lesion. Hence, the distance holder may especially be configured to provide a channel with a wall configured to provide that channel and configured to bridge a distance between the spray applicator and a topical area of a user, wherein the wall is further especially configured to circumferentially surround the spray during spraying of the treatment liquid. Hence, the wall may especially be configured to substantially allow the treatment liquid escaping from the distance holder only via the opening in the distance holder. Therefore, the distance holder may in embodiments essentially comprise a first opening configured to enclose a topical lesion and a second opening for entrance of the treatment liquid or at least part of the spray valve system, wherein the wall is configured to provide a channel with the first opening at one side and a second opening at an opposite side, wherein the wall may optionally include further openings, such as for influx of air. Especially the ratio of the cumulative (smallest) cross-sectional areas of these openings to the area of the wall less then 3:1, such as less than 2:1, like less than 1:1, or even less than 0.1:1. Here the term “smallest cross-sectional area” may be applied, as the openings in the wall may optionally have tapered shapes. The distance holder including said wall may especially have a tapered shape or a cylindrical shape, and may optionally consist of a transparent material, such as PMMA, siloxanes, etc.. Hence, in an embodiment the distance holder (120) includes a transparent part. Further, especially the distance holder may comprise the (first) opening and a second opening for entrance of the treatment liquid or at least part of the spray valve system, and a wall configured to provide a channel with the opening at one side and the second opening at an opposite side. Further, the wall may optionally include one or more openings.

In an embodiment, the treatment liquid may further comprises vitamin A. Vitamin A is a retinoid, which may act to affect HPV induced lesions to prevent recurrence of the virus. Vitamin A may evoke or potentiate immune response to warts and thus is combination with the described composition, treatment may be more effective when vitamin A is included. Hence, vitamin A may promote healing and/or prevent reoccurrence of the skin lesion, especially warts.

In yet a further aspect, the invention also provides a method of providing the spray applicator, especially as defined herein, the method comprising:
premixing acid and an aqueous liquid and providing the mixture to the container;
providing the propellant in a liquid phase to the container; and
closing the container with the spray valve system (and arrange a manual applicator to the spray valve system).

Further, the invention provides a kit of parts with comprising a container with a spray valve system as defined herein, and a distance holder, especially a plurality of distance holders having differently sized openings. Each of the distance holders is especially configured to be functionally coupled to the container with spray valve system to provide said spray applicator. The kit of parts may further comprise a patch comprising a porous part with pores. The treatment liquid, especially under pressure, may penetrate the porous part and come into contact with the skin lesion. The patch may be a further protection means and/or dosage means.

The term “substantially” herein, such as in “substantially consists”, will be understood by the person skilled in the art. The term “substantially” may also include embodiments with “entirely”, “completely”, “all”, etc. Hence, in embodiments the adjective substantially may also be removed. Where applicable, the term “substantially” may also relate to 90% or higher, such as 95% or higher, especially 99% or higher, even more especially 99.5% or higher, including 100%. The term “comprise” includes also embodiments wherein the term “comprises” means “consists of”. The term “and/or” especially relates to one or more of the items mentioned before and after “and/or”. For instance, a phrase “item 1 and/or item 2” and similar phrases may relate to one or more of item 1 and item 2. The term “comprising” may in an embodiment refer to “consisting of” but may in another embodiment also refer to “containing at least the defined species and optionally one or more other species”.

Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

The devices herein are amongst others described during operation. As will be clear to the person skilled in the art, the invention is not limited to methods of operation or devices in operation.

It should be noted that the above-mentioned embodiments illustrate rather than limit the invention, and that those skilled in the art will be able to design many alternative embodiments without departing from the scope of the appended claims. In the claims, any reference signs placed between parentheses shall not be construed as limiting the claim. Use of the verb “to comprise” and its conjugations does not exclude the presence of elements or steps other than those stated in a claim. The article “a” or “an” preceding an element does not exclude the presence of a plurality of such elements. The invention may be implemented by means of hardware comprising several distinct elements, and by means of a suitably programmed computer. In the device claim enumerating several means, several of these means may be embodied by one and the same item of hardware. The mere fact that certain measures are recited in mutually different dependent claims does not indicate that a combination of these measures cannot be used to advantage.

The invention further applies to a device comprising one or more of the characterizing features described in the description and/or shown in the attached drawings. The invention further pertains to a method or process comprising one or more of the characterising features described in the description and/or shown in the attached drawings.

The various aspects discussed in this patent can be combined in order to provide additional advantages. Furthermore, some of the features can form the basis for one or more divisional applications.

### BRIEF DESCRIPTION OF THE DRAWINGS

Embodiments of the invention will now be described, by way of example only, with reference to the accompanying schematic drawings in which corresponding reference symbols indicate corresponding parts, and in which:

FIGS. 1a-1d schematically depicts embodiments and aspects of the spray applicator.

The drawings are not necessarily on scale.

### DETAILED DESCRIPTION OF THE EMBODIMENTS

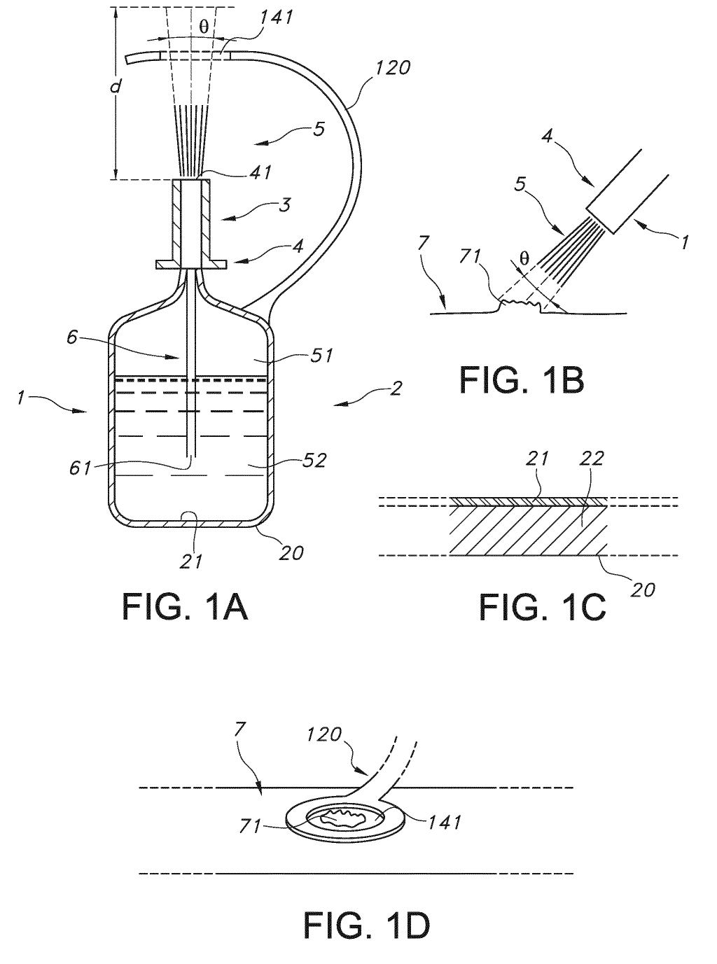

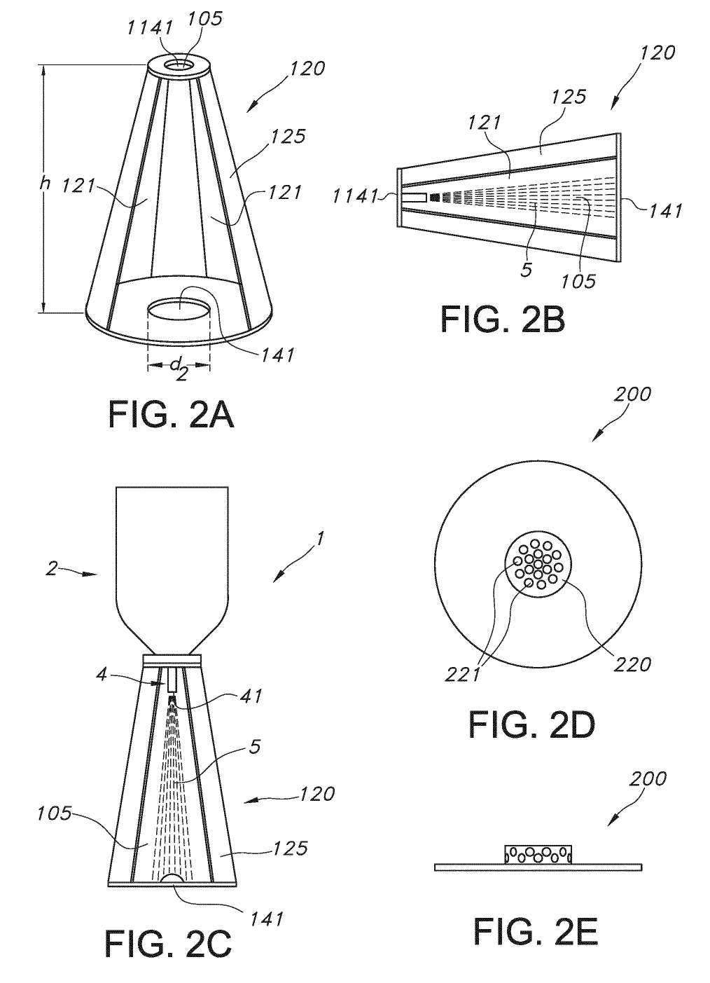

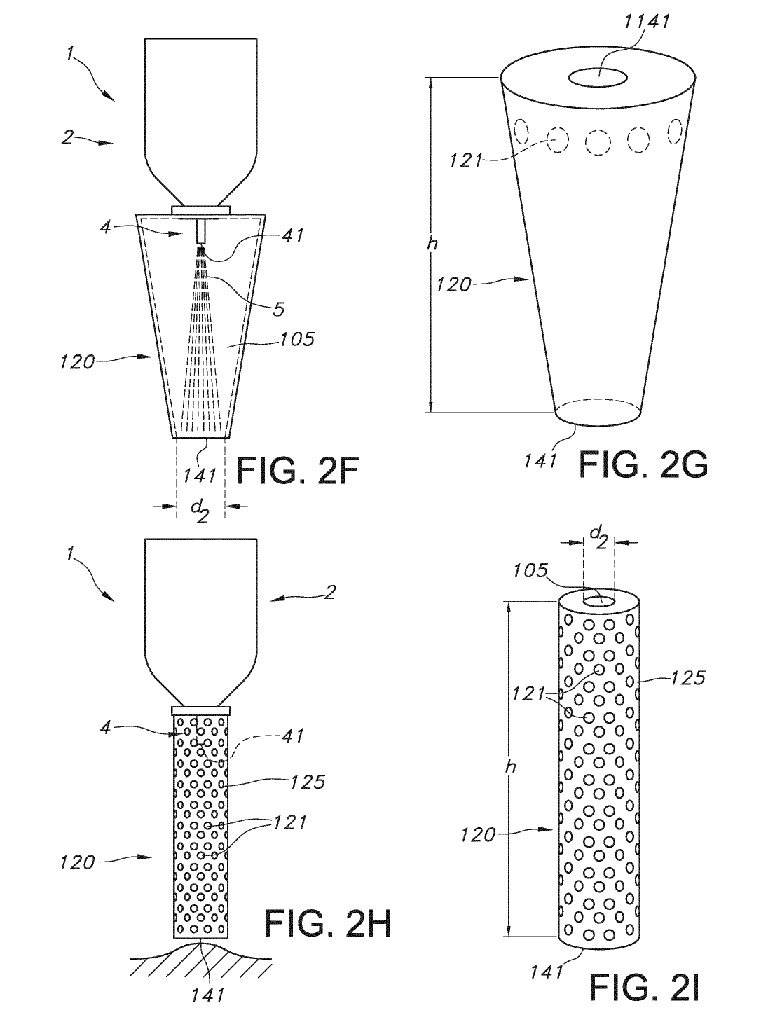

FIG. 1a schematically depicts a embodiment of the spray applicator 1. The spray applicator is especially an aerosol spray applicator. The spray applicator 1 comprises a can or container 2 as well as a spray valve system 4 with a nozzle or outlet 41. The container 2 comprises a container wall 20, which may optionally include a container wall coating 21. Further, optionally integrated with the valve system 4, the spray system 4 comprises a manual applicator. For instance by pressing this manual applicator, the spray valve systems frees treatment liquid 52 from the container 2 which is under elevated pressure due to the presence of a propellant under pressure. The spray valve system 4 has an opening or nozzle 41. The propellant is indicated with reference 51. Part of the propellant may also be in the liquid phase. The spray applicator may further comprise a dip tube 6, in fluid connection with the spray valve system 4 and with a dip tube opening 61, which during most of the lifetime of the spray applicator 1 will be emerged in the treatment liquid.

In an embodiment, the spray applicator 1 may especially be configured to provide a single spray dose of not more than 0.75 ml. Further, the spray applicator 1 may be configured to provide a spray 5 having a spray angle θ wherein 90% of a spray dose escapes from an outlet 41 of the spray valve system 4, wherein the spray angle θ is in the range of 15-45°. These spray characteristics may especially be obtained within a distance d of the nozzle 41, such as within a distance of 0-5 cm, like 0.5-5 cm, such as 0.5-2 cm. The spray valve system may include a metering valve. Especially, the spray valve system 4 is configured to administer the treatment liquid in a metered manner.

Optionally, the spray applicator 1 may include a distance holder 120, which may further include an opening 141 (herein also indicated as “first opening”). With such applicator, the opening may be arranged around the wart, and the jet may only provided to the topical area of the opening (see also FIG. 1d). Optionally, the distance holder includes transparent regions, such as openings or transparent material, thereby facilitating the user to easier perceive the topical area that is (to be) treated.

FIG. 1a is a very schematic embodiment. Of course, the spray applicator may include a container in a holder. Such holder may for instance host the optional distance holder 120. The distance holder 120 may be integrated in the spray applicator 1 or may be temporarily connected to the spray applicator. Optionally the distance holder 120 is bendable and can be arranged in a position for use and a position for rest. The former position may e.g. provide a smaller spray applicator (for transport purposes).

In the schematic drawing of 1a, the distance holder 120 does not entirely circumferentially surround the spray 5 during application.

FIG. 1b schematically depicts how the spray applicator 1 can be used. For the sake of simplicity, only the valve system 4 with nozzle 41 is shown. The spray or spray het 5 may be applied to a lesion 71, such as a wart, on a body surface or topical area 7, such as the skin.

FIG. 1c schematically depicts part of the container wall of the container. The container wall 20 may optionally comprise a container wall coating 21. This coating will thus be in contact with the treatment liquid. A top layer of the container wall 20 is indicated with reference 22. The container wall may e.g. be of aluminum or (stainless) steel and thus the top layer 22 is of aluminum or (stainless) steel. On the top layer 22, optionally the coating 21 may be available, for e.g. a PP coating.

FIG. 1d schematically depicts how the distance holder 120 can be arranged to a topical region or body surface 7, such as the skin, with the skin lesion 71, such as a wart surrounded by the opening 141. This allows dosing the treatment liquid to the opening only, thus the topical region in the opening only, and substantially not to healthy topical regions. Optionally, the opening is scalable, i.e. the opening area may be adapted by the user. For instance a diaphragm type of opening may be used.

FIGS. 2a-2c and 2f-2i schematically depict embodiments of distance holders 120 that substantially surround a spray during use. Referring to FIGS. 2a-2c the distance holder has a conical shape with a first opening 141 configured to enclose a topical lesion. Further, the distance holder comprise a second opening 1141 configured to receive at least part of the spray valve system, especially at least to circumferentially surround the opening or nozzle 41 of the spray valve system 4. Reference 125 indicates the wall of the distance holder 125 and reference 121 indicates the opening(s) in the wall of the distance holder 120. Especially the ratio of the cumulative (smallest) cross-sectional areas of these openings to the area of the wall less then 3:1, such as less than 2:1, like less than 1:1, or even less than 0.1:1. For instance, referring to the cone of FIG. 2a, the (external) area of the openings 121 relative to the wall (parts) 125 may be in the range of about 2:1. Reference 5 indicates the channel that is provided by wall 125 and through which the spray may be provided. The distance holder may have a height h in the range of 0.5-10 cm, such as especially 0.5-5 cm, such as 1-5 cm or for instance 0.5-2 cm. The first opening 141 is schematically depicted as circularly shaped. However, the shape may also be different. The diameter of the opening is indicated with reference d2. The equivalent circular diameter, i.e. the diameter as if the first opening is circular, may be in the range of 0.2-5 cm, such as in the range of 0.5-4 cm.

FIGS. 2d-2e schematically depict a patch 200 that may be configured over the topical area, with a porous part 220 with pores 221 over the topical lesion. The pores 221 are configured to allow penetration of the treatment liquid. FIG. 2e is a side view of an embodiment. Hence, in an embodiment, plasters or other patches, with e.g. a sponge membrane, may be applied to apply the liquid to the topical lesion. The treatment liquid is provided through the porous part, such as a membrane, like a sponge membrane. With this embodiment, the spray applicator may be used with or without distance holder 120, especially with the distance holder 120. When using the distance holder, the opening 141 of the distance holder can be placed on the patch 200 over at least part of the porous part 220.

FIGS. 2f-2g schematically depicts a distance holder 120 similar to the distance holder schematically depicted in FIGS. 2a-2c. However, now the distance holder has conical shape which is opposite. Further, here the distance holder 120 has a wall 125 that is closed, though in FIG. 2g optional openings 121 are depicted. Such openings 121 may be configured closer to the second inlet 1141 than to the first inlet 141.

FIGS. 2h-2i schematically depict an embodiment of the distance holder having substantially cylindrical shaped wall 125, with a plurality of relative small openings 125.

In a further embodiment, the invention also provides a kit of parts comprising a container 2 with a spray valve system 4 and a plurality of distance holders 120 having differently sized (first) openings 141. Each distance holders 120 can functionally be coupled to the container with spray valve system to provide the above described spray applicator 1. With different sized openings, for instance to be used depending on wart size, dosage may also be controlled. The size of the opening 141 may also that determine how many sprays of a treatment liquid should be administered. Referring to FIGS. 2a-2c and 2f-2i, optionally the first opening 141 is adjustable in size. Hence, the invention provides amongst others a composition for treatment of skin lesions by using a special designed (1) coated aluminum or (stainless) steel container, (2) a dosage delivery valve and nozzle, (3) a targeted applicator, (4) an active medical serum (acid+freezing agent), and optionally (5) a child safety lock. For instance, a composition for treatment of superficial lesions may be provided, wherein a freezing agent is applied having a low boiling point and an (extreme) skin exfoliating acid, such as acetic acid. Further, the invention provides a device for the application of the active serum on the skin lesion, wherein the device comprises an applicator to include a dosage delivery nozzle to apply, in an acid compliant coated aluminum or (stainless) steel container consisting of a (tubular) container and (cylindrical) body with a targeted open applicator which ensures that the dosage valve is released at a certain distance from the skin to allow oxidization for controlled temperature of the freezing agent, along with the active serum to treat the skin lesion. TCA may advantageously be provided as powder, which can be combined with a liquid to provide the treatment liquid.

Experimental work was done wherein the effectivity of acids on warts, the effectivity of cryogenic treatment of warts, and the effectivity of the combination of acids and cryogenic treatment of warts in one dosage, as described above, was evaluated.

TABLE 1
Analysis of combination therapy of 30% TCA with cryogen

| | | Weeks to | Applications of 30% TCA |
|---|---|---|---|
| Sex | Age | Resolution | with cryogen (−57° C.) |
| F | 10 | 4 | 4 |
| M | 11 | Unresolved | 17 |
| M | 7 | 3 | 3 |
| M | 7 | 3 | 3 |
| M | 7 | 3 | 3 |
| M | 7 | 3 | 3 |
| M | 7 | 3 | 3 |
| F | 8 | 9 | 9 |
| M | 15 | 8 | 7 |
| M | 15 | 8 | 7 |
| M | 15 | 8 | 7 |
| M | 15 | 16 | 15 |
| M | 15 | Unresolved | 20 |
| F | 12 | 3 | 3 |
| F | 12 | 2 | 3 |
| F | 12 | 2 | 3 |
| F | 12 | 7 | 3 |
| F | 12 | 7 | 3 |
| M | 8 | 16 | 11 |
| F | 8 | 8 | 7 |
| F | 8 | 8 | 7 |
| F | 6 | 6 | 5 |
| F | 6 | 6 | 5 |
| F | 6 | 6 | 5 |
| M | 51 | 3 | 3 |
| F | 15 | 9 | 8 |

Of the 28 treated warts, 93% resolved successfully via this combination method over an average of 5.4 weeks and 5.96 acid-cryogen applications. The combined cryogen/acid treatment method can be seen as a low-risk, efficient, gentle and highly successful method to treat warts (and other topical lesions). The results are inline with the assumptions of combining the two modes of action of an acid (peeling of the skin) and a cryogen (destruction of HPV infected cells).

1. A spray applicator for topical treatment, the spray applicator comprising a container comprising a treatment liquid under pressure and a propellant, wherein the container is in fluid connection with a spray valve system for application of at least part of the treatment liquid on a topical area, wherein the treatment liquid comprises an acid, wherein the spray applicator further comprises a distance holder configured to arrange on a topical area and comprising a opening configured to enclose a topical lesion, wherein the spray applicator is configured to spray the treatment liquid through the opening in the distance holder.

2. The spray applicator according to claim 1, wherein the treatment liquid at least comprises trichloro acetic acid.

3. The spray applicator according to claim 1, wherein
the acid comprises one or more acids selected from the group consisting of trichloro acetic acid, salicylic acid, formic acid, glycolic acid, dichloro acetic acid, monochloro acetic acid, boric acid, nitric acid, sulfuric acid, phosphoric acid, oxalic acid, lactic acid, hydrochloric acid,
the propellant comprises one or more propellants selected from the group consisting of dimethyl ether (DME), diethyl ether (DEE), methyl ethyl ether (methoxy ethane), carbon dioxide (CO₂), nitrogen (N₂), nitrous oxide, propane, butane, liquified petroleum gases (LPG), and a chlorofluorcarbon (CFC),
wherein the container comprises a container wall comprising aluminum or (stainless) steel and a coating comprising one or more of a polymer and a resin, wherein the top layer coating comprises one or more of polyethylene and polypropylene; and
wherein the metal of the top layer of the wall comprises aluminum or (stainless) steel.

4. The spray applicator according to claim 1, wherein the treatment liquid comprises at least trichloroacetic acid (TCA) and wherein the propellant comprises at least one or more of dimethyl ether (DME), diethyl ether (DEE), and methyl ethyl ether (methoxy ethane).

5. The spray applicator according to claim 1, wherein the spray valve system includes an infant opening protection.

6. The spray applicator according to claim 1, wherein the propellant has a boiling point of less than 10° C., and wherein pressure in the container is in the range of at least 5 bar.

7. The spray applicator according to claim 1, wherein the treatment liquid comprises the acid in a concentration up to 40 wt. %, especially wherein the treatment liquid comprises the acid in a concentration in the range of 1-10 wt. %.

8. The spray applicator according to claim 1, wherein the spray applicator is configured to provide a single spray dose of not more than 0.75 ml.

9. The spray applicator according to claim 1, wherein distance holder includes a transparent part.

10. The spray applicator according to claim 1, wherein the distance holder comprise said opening and a second opening for entrance of the treatment liquid or at least part of the spray valve system, and a wall configured to provide a channel with the opening at one side and the second opening at an opposite side.

11. The spray applicator according to claim 10, wherein the wall includes one or more openings.

12. The spray applicator according to claim 1, wherein the distance holder is configured to prevent physical contact between the spray valve system and the topical area, wherein the opening is adjustable in size.

13. The spray applicator according to claim 1, wherein the spray applicator is configured to provide a spray having a spray angle wherein 90% of a spray dose escapes from an outlet of the spray valve system, wherein the spray angle is in the range of 15-45°.

14. The spray applicator according to claim 1, wherein the treatment liquid further comprises vitamin A.

15. Use of the spray applicator according to claim 1 for a cosmetical treatment of a topical lesion selected from the group consisting of epidermodysplasia veruciformis, a HPV caused skin disorder, acne, a scar, a wrinkle, a tattoo, eyelid xanthelasma, an age spot, a senial lentigo, a solar lentigo, a melasma, a hand wart, a plantar wart, a corn, an ingrown toe nail, a mucous membrane lesion.

16. Use of the treatment liquid as defined in claim 1, for a cosmetical treatment of a topical lesion selected from the group consisting of epidermodysplasia veruciformis, a HPV caused skin disorder, acne, a scar, a wrinkle, a tattoo, eyelid xanthelasma, an age spot, a senial lentigo, a solar lentigo, a melasma, a hand wart, a plantar wart, a corn, an ingrown toe nail, a mucous membrane lesion.

17. A composition comprising the treatment liquid and the propellant as defined in claim 1, for use in freezing and acid treatment of a topological lesion.

18. The composition according to claim 17, for use in the treatment of a wart or a corn.

19. The composition according to claim 17, wherein the composition further comprises vitamin A.

20. A method of providing the spray applicator according to claim 1, the method comprising:
premixing acid and an aqueous liquid and providing the mixture to the container;
providing the propellant in a liquid phase to the container; and
closing the container with the spray valve system.

21. A kit of parts comprising a container with a spray valve system as defined in claim 1, and a plurality of distance holders having differently sized openings.

22. The kit of parts according to claim 21, further comprising a patch comprising a porous part with pores.
